(19) United States
(12) Patent Application Publication
(10) Pub. No.: US 2008/0234640 A1
(43) Pub. Date: Sep. 25, 2008

(54) Needle Arrangement for a Low Vacuum Wound Drainage System

(76) Inventor: Deon Juan Fourie, Centurion (ZA)

Correspondence Address: NIXON PEABODY, LLP, 401 9TH STREET, NW, SUITE 900, WASHINGTON, DC, 20004-2128, US

(21) Appl. No.: 12/064,573

(22) PCT Filed: Aug. 25, 2006
(86) PCT No.: PCT/IB2006/002312

§ 371 Date: Feb. 22, 2008

(30) Foreign Application Priority Data
Aug. 25, 2005 (ZA) 2005/06821

(51) Int. Cl. A61M 5/32

(52) U.S. Cl. 604/272

(57) ABSTRACT

The invention provides a needle arrangement (30) for a low-vacuum wound drainage system. The needle arrangement (30) includes a needle (32) and a needle retriever (34). The needle (32) has a sharpened, leading end (36) for piercing the skin of a patient, and a first locking formation (42) proximate the leading end of the needle. The needle retriever (34) has a second locking formation (48) for engaging the first locking formation (42) on the needle (32) when the needle retriever (34) is located over the leading end (36) of the needle (32), thereby to lock the needle retriever (34) to the needle, and a handle (52) for allowing the needle retriever (34) to be held and pulled by a user, thereby to retrieve the needle (32) from the patient.

### BACKGROUND OF THE INVENTION

THIS invention relates to medical equipment, and more specifically to low-vacuum wound drainage systems. In particular, the invention relates to a needle arrangement for inserting a tube for a low-vacuum wound drainage system into a wound of a patient.

Generally, low-vacuum wound drainage systems include a bellows-type drainage container which is connectable to a wound of a patient by means of one or more flexible tubes. The drainage container usually is collapsible and expandable bellows fashion, and is biased into an expanded condition by a compression coil spring. When the container is collapsed against the bias of the spring, sealed and connected to the flexible tube, it serves to suck or draw body fluids out of the wound as the spring biases the drainage container towards the expanded condition.

Conventionally, the flexible tube is inserted into a patient's wound by placing a needle connected to the tube into the wound prior to stitching the wound closed, puncturing the patient's skin from within the wound, and drawing the needle out of the wound through the patient's skin until an end section of the tube including openings along its length is located within the open wound. Thereafter, the wound is stitched closed, and the flexible tube is disconnected from the needle for connection to the drainage container, typically via an adapter and another length of flexible tube. A valve may be located on one of the lengths of flexible tube for regulating the flow of fluid to the drainage container.

A disadvantage associated with conventional needles of the type described above is that they tend to be relatively difficult to grip when attempting to fully withdraw the needle from the wound. This is largely due to the fact that these needles are smooth and, when covered with blood and other body fluids, tend to be slippery.

It is an object of the present invention to provide a needle arrangement for a low-vacuum wound drainage system which is relatively easy to withdraw from a patient's wound.

### SUMMARY OF THE INVENTION

According to the invention there is provided a needle arrangement for a low-vacuum wound drainage system, the needle arrangement comprising:
a needle which includes:
   a sharpened, leading end for piercing the skin of a patient; and
   a first locking formation proximate the leading end of the needle; and
a needle retriever which includes:
   a second locking formation for engaging the first locking formation on the needle when the needle retriever is located over the leading end of the needle, thereby to lock the needle retriever to the needle; and
   a handle for allowing the needle retriever to be held and pulled by a user, thereby to retrieve the needle from the patient.

In a preferred embodiment of the invention, the first locking formation comprises at least one locking recess, and the second locking formation comprises one or more locking projections which are engagable with the at least one locking recess to lock the needle retriever to the needle.

In a particularly preferred arrangement, the first locking formation comprises an annular locking recess, and the needle retriever includes a sheath for receiving the leading end of the needle and one or more locking projections on the sheath for engaging the locking recess.

The retriever may include a protective flange adjacent the handle for protecting a user's hand, in use.

In one embodiment, the retriever comprises a first component which includes the handle, a second component which includes the second locking formation, and connecting means for connecting the first and second components together. With this embodiment, a user may connect the first component of the retriever to one of several different second components having differently configured second locking formations.

The connecting means may comprise a connecting formation on the first component which is engagable with a corresponding connecting formation on the second component. For example, the second component may include one or more projections which are arranged to clip into one or more recesses in the first component.

Typically, the needle includes a kink or bend along its length to facilitate the use thereof.

The needle may also include engaging formations at or near a trailing end, opposite the leading end, for engaging a tube or the like, in use.

The invention extends to a needle for a low-vacuum wound drainage system, the needle comprising:
a sharpened, leading end for piercing the skin of a patient; and
a locking formation proximate the leading end of the needle, the locking formation being engagable with a corresponding locking formation on a needle retriever so as to lock the needle to the needle retriever when the needle retriever is located over the leading end of the needle.

The invention also extends to a needle retriever for a low-vacuum wound drainage system, the needle retriever comprising:
a body for at least partially covering a leading, sharpened end of a needle for the low-vacuum wound drainage system;
a locking formation for engaging a corresponding locking formation on the needle when the needle retriever is located over the leading end of the needle, thereby to lock the needle retriever to the needle; and
a handle for allowing the needle retriever to be held and pulled by a user, thereby to retrieve the needle from a patient.

### BRIEF DESCRIPTION OF THE DRAWINGS

The invention will now be described in more detail, by way of example only, with reference to the accompanying drawings in which:

FIG. 1 shows, diagrammatically, a low-vacuum wound drainage system for draining body fluids from a wound of a patient;

FIG. 2 illustrates a perspective view of a needle arrangement according to the present invention for inserting a flexible tube into a patient's wound;

FIG. 3 illustrates a side view of the needle arrangement of FIG. 2 with a needle retriever detached from a needle;

FIG. 4 illustrates a side view similar to that of FIG. 3 during attachment of the needle retriever to the needle;

FIG. 5 illustrates a side view similar to that of FIG. 4 with the needle retriever attached to the needle;

FIG. 6 illustrates an exploded perspective view of a second embodiment of the needle retriever comprising two separate components;

FIG. 7 illustrates the needle retriever of FIG. 6 with the two needle retriever components connected together;

FIG. 8 illustrates a partial cross-sectional view of the two needle retriever components in an unassembled form; and

FIG. 9 illustrates a partial cross-sectional view of the two needle retriever components in an assembled form.

### DETAILED DESCRIPTION OF THE INVENTION

The present invention has application in the medical field, and more particularly in low-vacuum wound drainage systems. The invention is embodied in a needle and a needle retriever which facilitate the insertion of a tube into a wound of a patient, and which facilitate the disposal of the needle after use.

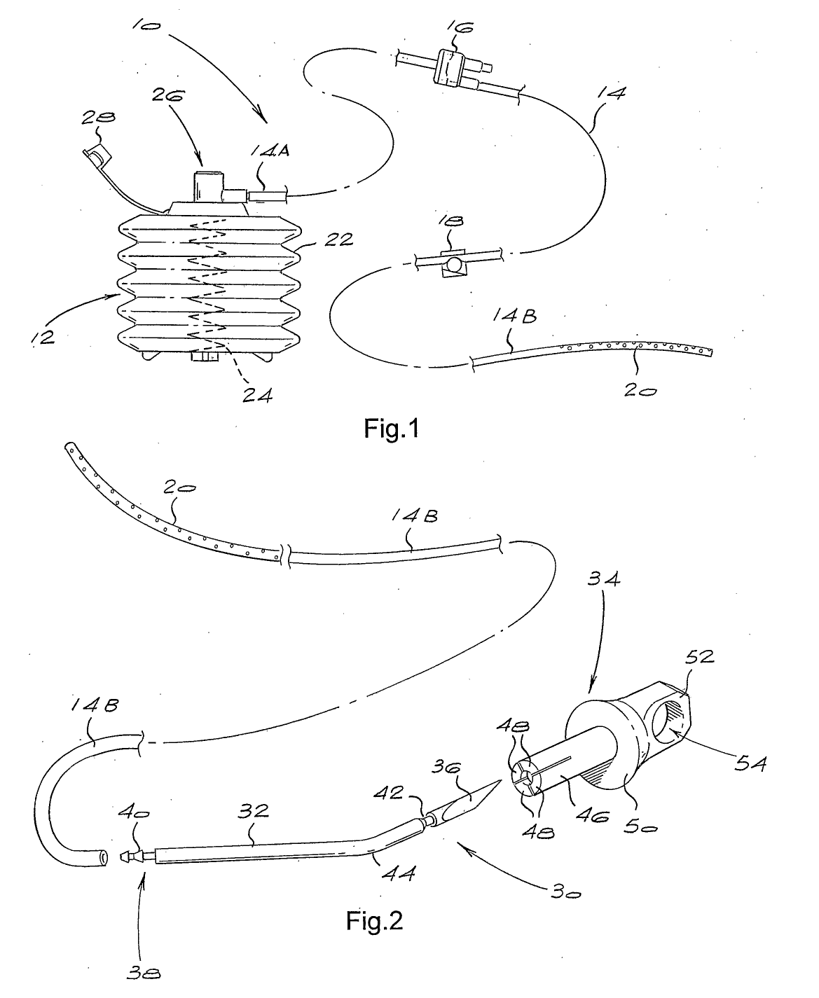

FIG. 1 of the accompanying drawings illustrates a low-vacuum wound drainage system for draining body fluids from a patient's wound. The system is designated generally with the reference numeral 10 and includes a bellows-type drainage container 12 which is connectable to a wound of a patient by means of tubing 14. Typically, the tubing 14 is formed from two lengths of flexible tube 14A and 14B, each of which has an internal diameter of 3 mm, 4 mm or 6 mm, depending on the application. The two lengths of tube 14A and 14B are connected together with an adapter 16, and a valve 18 may be provided for regulating the flow of fluid through the tubing 14. A section 20 of the tubing 14 defines a plurality of openings along its length for allowing fluids into the tubing 14.

The container 12 is conventional in nature and hence will not be described in detail. Generally, however, and container 12 includes a flexible outer wall 22 which is formed from a plastics material and which is collapsible and expandable bellows fashion. A compression coil spring 24 is located inside the container 12 and is arranged to bias the outer wall 22 of the container into the expanded condition illustrated in FIG. 1. The container 12 includes a drainage opening 26 for allowing fluid to be discharged from the container, and a closure 28 for hermetically sealing the drainage opening 26 when inserted into this opening.

With reference now to FIGS. 2 to 5 of the accompanying drawings, a needle arrangement according to the present invention is designated generally with the reference numeral 30. The needle arrangement 30 includes a needle 32 and a needle retriever 34. The needle 32, which may be formed from stainless steel, has a sharpened, leading end 36 for puncturing the skin of a patient, and a trailing end 38 which defines a formation 40 for engaging the tubing 14. The needle 32 also includes a locking formation in the form of an annular locking recess 42 which is located proximate the leading end 36, as shown. A kink or bend 44 between the locking recess 42 and the trailing end 38 facilitates the use of the needle.

With particular reference now to FIG. 2 of the drawings, the needle retriever 34 may include a sheath 46 which is designed to cover the leading end 36 of the needle 32, in use. In the illustrated embodiment, a locking formation in the form of four projections 48 extends radially inwardly, as shown. The projections 48 are designed to engage the locking recess 42 in a manner which is described in more detail below so as to lock the needle retriever 34 to the needle 32.

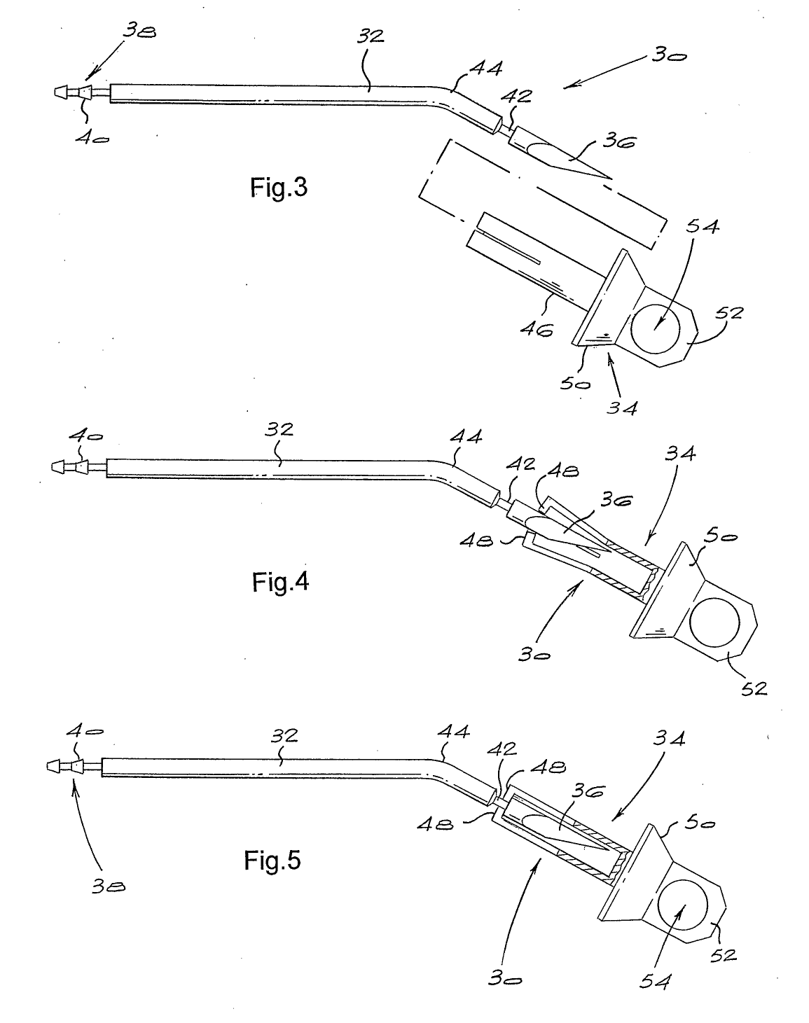

Referring also to FIGS. 3 to 5 of the drawings, the needle retriever 34 includes a protective flange 50 adjacent a handle 52. The handle defines an opening 54 which facilitates gripping of the handle, in use. Typically, the retriever 34 is formed from a resilient plastics material such as polyethylene or polypropylene.

In practice, the needle 32 and the retriever 34 are used to insert one end of the tubing 14 into a wound of a patient as follows. First, the needle 32 is connected to the flexible tube 14B with the section 20 distal from the needle. Thereafter, the leading end 36 of the needle 32 is located in an open wound of a patient and is forced through the patient's skin so as to exit the wound. Once the leading end 36 has emerged from the patient's skin, the needle retriever 34 may be used to retrieve the needle 32. For this purpose, the handle 52 of the needle retriever 34 is gripped in the user's hand so that the user's fingers are located behind the protective flange 50, and the sheath 46 is carefully pressed over the leading end 36 of the needle 32 so that the projections 48 ride along the needle in the manner illustrated in FIG. 4. When the locking projections 48 reach the locking recess 42, the resiliency of the sheath 46 causes the locking projections to clip into the locking recess, thereby locking the needle retriever 34 to the needle 32 (see FIG. 5). The handle 52 of the needle retriever 34 may then be pulled to withdraw the needle from the patient's body. Typically, the needle 32 is withdrawn from the patient until the section 20 of the flexible tube 14B is located within the patient's wound, at which point the tube 14B may be cut adjacent the needle 32 so as to detach the needle from the tube, and the wound may be stitched closed. The cut end of tube 14B may then be connected to the adapter 16 in the manner illustrated in FIG. 1.

With the container 12 collapsed and the closure 28 located in the drainage opening 26, suction generated by the biasing of the container wall 22 towards the expanded condition causes body fluids generated within the wound to be drawn along the tubing 14 and into the container 12. It will be appreciated that the valve 18 may then be used to regulate the flow of body fluid to the container 12.

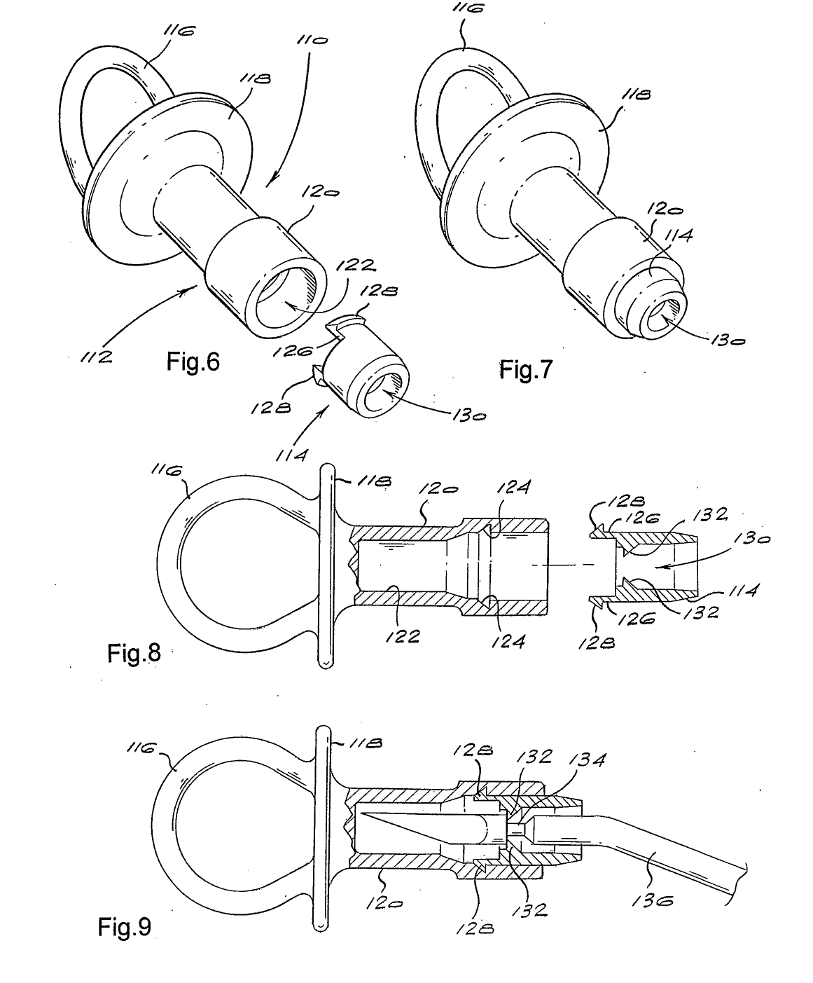

FIGS. 6 to 9 of the accompanying drawings illustrate a needle retriever according to a second embodiment of the invention. In this embodiment, the needle retriever 110 includes a first component 112 and a second component 114. The first component 112 has a handle 116 on one side of a protective flange 118, and a sleeve 120 defining a socket 122 on the other side of the flange 118. With particular reference to FIG. 8, the socket 122 defines a connecting formation in the form of an annular groove or recess 124.

The second component 114 includes a connecting formation in the form of a pair of connecting lugs 126, each of which includes a clipping projection 128 for clipping into the recess 124 in the first component 112. The second component 114 also defines a passage 130 including locking formations in the form of projections 132 which are arranged to engage with a locking groove 134 in the leading end of a needle 136 in the manner illustrated in FIG. 9. The locking formations 132 may comprise two or more projections which are evenly spaced around the circumference of a portion of the passage 130. Preferably, several different second components 114 are provided with differently configured locking formations 132 so as to accommodate needles of different sizes.

In practice, a user may select a particular second component 114 which includes locking formations 132 of a desired configuration, and this second component 114 may then be connected to the first component 112 by simply inserting the second component into the socket 122 of the first component until the clipping projections 128 on the connecting lugs 126 clip into the recess 124 in the first component to lock the second component 114 to the first component 112. Thereafter, the needle retriever 110 may be used to engage and withdraw a needle from a patient's wound in a similar fashion to that described above with reference to the first embodiment of the invention.

One advantage of the needle arrangement according to the present invention is that the needle retriever 34, 110 facilitates the withdrawal of the needle 32, 136 from the patient's wound by providing a mechanism for easily gripping the needle 32, 136. It should also be understood that the protective flange 50, 118 protects the user's fingers from a needle stick injury during attachment of the needle retriever 34, 110 to the needle 32, 136. Furthermore, once the needle retriever 34, 110 has been located over the leading end of the needle 32, 136, it serves to safely cover the needle point for disposal of the needle. Accordingly, the needle may be disposed of after use without any further procedure.

1. A needle arrangement for a low-vacuum wound drainage system, the needle arrangement comprising:
a needle which includes:
a sharpened, leading end for piercing the skin of a patient; and
a first locking formation proximate the leading end of the needle; and
a needle retriever which includes:
a second locking formation for engaging the first locking formation on the needle when the needle retriever is located over the leading end of the needle, thereby to lock the needle retriever to the needle; and
a handle for allowing the needle retriever to be held and pulled by a user, thereby to retrieve the needle from the patient.

2. A needle arrangement according to claim 1, wherein the first locking formation comprises at least one locking recess, and the second locking formation comprises one or more locking projections which are engagable with the at least one locking recess to lock the needle retriever to the needle.

3. A needle arrangement according to claim 1, wherein the first locking formation comprises an annular locking recess, and the needle retriever includes a sheath for receiving the leading end of the needle and one or more locking projections on the sheath for engaging the locking recess.

4. A needle arrangement according to claim 1, wherein the retriever includes a protective flange adjacent the handle for protecting a user's hand, in use.

5. A needle arrangement according to claim 1, wherein the retriever comprises a first component which includes the handle, a second component which includes the second locking formation, and connecting means for connecting the first and second components together.

6. A needle arrangement according to claim 5, including more than one needle retriever second component, wherein each of the needle retriever second components is connectable to the needle retriever first component, and wherein the configuration of the second locking formation on each of the needle retriever second components differs from that of the other needle retriever second components so that the needle retriever may accommodate needles of different sizes.

7. A needle arrangement according to claim 6, wherein the connecting means comprises a connecting formation on the first component which is engagable with a corresponding connecting formation on the second component.

8. A needle arrangement according to claim 7, wherein the needle retriever second component includes one or more projections which are arranged to clip into one or more recesses in the needle retriever first component.

9. A needle arrangement according to claim 1, wherein the needle includes a kink or bend along its length to facilitate the use thereof.

10. A needle arrangement according to claim 1, wherein the needle also includes engaging formations at or near a trailing end, opposite the leading end.

11. A needle for a low-vacuum wound drainage system, the needle comprising:
a sharpened, leading end for piercing the skin of a patient; and
a locking formation proximate the leading end of the needle, the locking formation being engagable with a corresponding locking formation on a needle retriever so as to lock the needle to the needle retriever when the needle retriever is located over the leading end of the needle.

12. A needle according to claim 11, including a kink or bend along its length to facilitate the use thereof.

13. A needle according to claim 11, including engaging formations at or near a trailing end, opposite the leading end.

14. A needle retriever for a low-vacuum wound drainage system, the needle retriever comprising:
a body for at least partially covering a leading, sharpened end of a needle for the low-vacuum wound drainage system;
a locking formation for engaging a corresponding locking formation on the needle when the needle retriever is located over the leading end of the needle, thereby to lock the needle retriever to the needle; and
a handle for allowing the needle retriever to be held and pulled by a user, thereby to retrieve the needle from a patient.

15. A needle retriever according to claim 14, including a protective flange adjacent the handle for protecting a user's hand, in use.

16. A needle retriever according to claim 14, comprising a first component which includes the handle, a second component which includes the locking formation, and connecting means for connecting the first and second components together.

17. A needle retriever according to claim 16, including more than one needle retriever second component, wherein each of the needle retriever second components is connectable to the needle retriever first component, and wherein the configuration of the locking formation on each of the needle retriever second components differs from that of the other needle retriever second components so that the needle retriever may accommodate needles of different sizes.

18. A needle retriever according to claim 17, wherein the connecting means comprises a connecting formation on the first component which is engagable with a corresponding connecting formation on the second component.

19. A needle retriever according to claim 18, wherein the needle retriever second component includes one or more projections which are arranged to clip into one or more recesses in the needle retriever first component.
